(19) United States
(12) Patent Application Publication
(10) Pub. No.: US 2006/0115462 A1
(43) Pub. Date: Jun. 1, 2006

(54) Direct DNA delivery to bone cells

(76) Inventors: Vladimir Subbotin, Madison, WI (US); Julia Hegge, Monona, WI (US)

Correspondence Address: MIRUS CORPORATION, 505 SOUTH ROSA RD, MADISON, WI, 53719, US

(21) Appl. No.: 11/286,719

(22) Filed: Nov. 23, 2005

Related U.S. Application Data
(60) Provisional application No. 60/632,145, filed on Dec. 1, 2004.

(51) Int. Cl. A61K 48/00

(52) U.S. Cl. 424/93.21; 514/44

(57) ABSTRACT

An method is described that enables in vivo delivery of polynucleotides to cells of a mammalian bone limb. The method involves the injection of polynucleotides in a large volume into the lumen of the bone.

### CROSS-REFERENCE TO RELATED APPLICATIONS

This application claims priority to Provisional Application No. 60/632,145, filed Dec. 1, 2004.

### BACKGROUND OF THE INVENTION

Gene therapy is the purposeful delivery of genetic material to cells for the purpose of treating disease or biomedical investigation and research. Gene therapy includes the delivery of a polynucleotide to a cell to express an exogenous nucleotide sequence, to inhibit, eliminate, augment, or alter expression of an endogenous nucleotide sequence, or to produce a specific physiological characteristic not naturally associated with the cell. In some cases, the polynucleotide itself, when delivered to a cell, can alter expression of a gene in the cell. A basic challenge in gene therapy is to develop approaches for delivering genetic information to different cells in vivo in a way that is efficient and safe. If genetic material are appropriately delivered they can potentially enhance a patient's health and, in some instances, lead to a cure. Delivery of genetic material to cells in vivo is also beneficial in basic research into gene function as well as for drug development and target validation for traditional small molecule drugs.

It was first observed that injection of plasmid DNA directly into muscle in vivo, enabled expression of foreign genes in the muscle (Wolff et al. 1990). Since that report, several other studies have reported the ability for foreign gene expression following the direct injection of DNA into the parenchyma of other tissues, including heart and liver. More recently, intravascular delivery of polynucleotides to tissues in vivo has been shown to be effective (Liu et al. 1999, Lewis et al. 2002, Budker et al. 1996, McCaffrey et al. 2002, Zhang et al. 1999, Budker et al. 1998, Zhang et al. 2001, Liu et al. 2001, Hodges et al. 2003, Eastman et al. 2002, Hagstrom et al. 2004). Intravascular delivery of polynucleotides has been further described in U.S. Pat. Nos. 6,265,387, 6,379,966, 6,627,616, 6,811,576, and 6,897,068 and in US Patent Publications US/2004/0136960, and US/2004/0242528. For intravascular gene delivery, vessel permeability and extravascular fluid volume is increased by one or more of the following: using a sufficient volume of an appropriate injection solution, injecting the solution at an appropriate rate and increasing permeability of the vessel wall.

We now describe an effective delivery method that enables in vivo delivery of polynucleotides to bone cells.

### SUMMARY OF THE INVENTION

In a preferred embodiment, we describe a method for in vivo delivery of polynucleotides to cells in bone comprising forming an access point in a bone wall, and injecting a solution containing the polynucleotides into the bone interior through the access point. Delivery of the polynucleotide to bone cells is facilitated by the injection volume and rate. Injecting a sufficient volume at a sufficient rate, determined by the size of the bone, increases the volume of extravascular fluid in the target tissue and movement of the polynucleotide into the target bone cells. The solution may additionally contain a compound or compounds which may or may not associate with the polynucleotide and may aid in delivery.

In a preferred embodiment, the cell may be selected from the group consisting of: osteocytes, osteoclasts, osteoblasts, bone marrow cells, and stromal cells.

The described method can be used to deliver a polynucleotide to a mammalian cell for the purpose of altering the endogenous properties of the cell, for example altering the endogenous properties of the cell for therapeutic purposes, for augmenting function, for facilitating pharmaceutical drug discovery, for facilitating drug target validation or for investigating gene function (i.e., research).

In a preferred embodiment, the process further comprises administration of at least one anesthetic or analgesic drug or adjuvant. Administration of anesthetics or analgesic lessens potential discomfort or pain experienced by the mammal during or after the procedure. Examples of such drugs included, not are not limited to: lidocaine, NSAIDs, clonidine, ketamine, neuromuscular blockers, and immunsuppressants.

Further objects, features, and advantages of the invention will be apparent from the following detailed description when taken in conjunction with the accompanying drawings.

### BRIEF DESCRIPTION OF THE DRAWINGS

FIG. 1. Diagram illustrating features in a section of bone.

FIG. 2. Illustration of a bone injection device:
A—hollow tube for inserting into bone, B—flexible tubing connected to a reservoir, C—microvise, D—elastic gasket with conical shape to seal the space between stainless steel tubing and bone tissue.

FIG. 3. Chart illustrating luciferase expression in bone following delivery of luciferase expression plasmid.

FIG. 4. Fluorescence micrograph of a bone section showing cells expressing yellow fluorescent protein. Left panel: F—actin staining. Right panel: Cells expressing yellow fluorescence protein. A—compact bone with osteocytes (arrows), B—periosteum (by structural definition).

FIG. 5. Fluorescence micrograph of a bone section showing cells expressing yellow fluorescent protein. Left panel—section immunostained for collagen. Right panel—same area showing yellow fluorescent protein expression. A—compact bone with very dense staining for collage, B—periosteum with low staining for collagen.

### DETAILED DESCRIPTION

Intraosseous infusions of fluid and drugs is a well accepted medical procedure. It was recognized in the early 1920s that the bone lumen can function as a noncollapsible vein, thereby providing a means for obtaining vascular access. Fluid injected into the bone lumen (medullary cavity) enters the venous system via venous sinusoids or canals within the lumen.

Bone is made up of a dense outer layer, compact tissue, surrounding a spongy inner layer that forms a meshwork occupied by bone marrow and fat tissue, cancellous tissue. The difference in structure between the compact tissue and the cancellous tissue depends upon the different amount of solid matter, and the size and number of spaces in each. The cavities are small in the compact tissue and the solid matter between them abundant, while in the cancellous tissue the spaces are large and the solid matter is in smaller quantity. Bone is permeated by vessels, and is enclosed, except where it is coated with articular cartilage, in a fibrous membrane, the periosteum, by means of which many of these vessels reach the hard tissue.

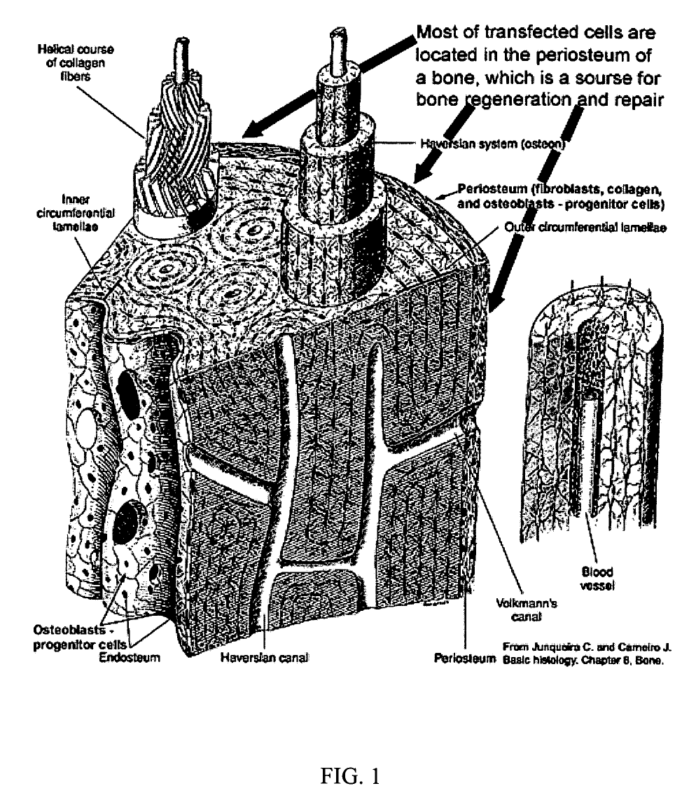

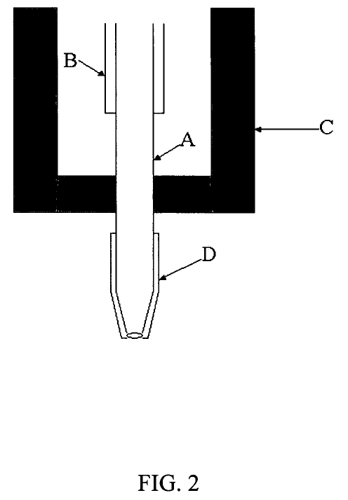

A transverse section of dense bone is observed to be mapped out into a number of circular districts each consisting of a central hole surrounded by a number of concentric rings (FIG. 1). These districts are termed Haversian systems; the central hole is a Haversian canal, and the rings are layers of bony tissue arranged concentrically around the central canal, termed lamellae. The canals run parallel with the longitudinal axis of the bone for a short distance and then branch and communicate. Near the medullary cavity the canals are larger than those near the surface of the bone. Each canal contains one or two blood vessels. Those canals near the surface of the bone open upon it by minute orifices, and those near the medullary cavity open in the same way into this space, so that the whole of the bone is permeated by a system of blood vessels running through the bony canals in the centers of the Haversian systems.

The blood vessels of bone are very numerous. The periosteum serves as a nidus for the ramification of the vessels previous to their distribution in the bone. From the periosteum, vessels pass into minute orifices in the compact tissue and run through the canals which traverse the compact tissue. The cancellous tissue is supplied in a similar way, but by less numerous and larger vessels, which, perforating the outer compact tissue, are distributed to the cavities of the spongy portion of the bone. Because of the structure of bone and its relationship with the venous system, substances injected into the lumen of a bone readily enter the venous system.

Adaptation of the intraosseous infusion method enables genetic material to be delivered to bone cells. To deliver polynucleotide to bone cells, a means for injection fluid into the bone, an injection device, is inserted into the target bone. A solution containing the polynucleotide is then injected into the interior of the bone under pressure. In one embodiment, a hole is drilled into the target bone using an appropriate surgical drill. A rigid hollow tube is positioned into the hole and the solution is injected into the bone through the tube. The tip of the tube, or other equivalent device, is advanced into the bone wall such that it terminates in the bone lumen but does not reach the opposite internal bone surface. An elastic gasket around the tube end which is pressed into the hole prevents leakage through the hole during the injection, allowing the solution to be injected under pressure. The pressure is sufficient to cause extravasation of the fluid and its contents from veins near the injected bone.

The injection volume and injection rate are dependent upon the size of the bone into which the solution is injected. Larger injection volumes and/or higher injection rates are required for larger target sizes. Consequently, for delivery in larger animals, injection of larger volumes is expected. The precise volume, or dose, for polynucleotide delivery to a particular bone may be determined empirically. By increasing the amount of polynucleotide injected and the volume of injection, the method described for delivery of polynucleotides to bone cells in small mammals such as mice and rats is readily adapted to use in larger animals.

The polynucleotide is injected in a pharmaceutically acceptable solution. Pharmaceutically acceptable refers to those properties and/or substances which are acceptable to the mammal from a pharmacological/toxicological point of view. The phrase pharmaceutically acceptable refers to molecular entities, compositions and properties that are physiologically tolerable and do not typically produce an allergic or other untoward or toxic reaction when administered to a mammal. Preferably, as used herein, the term pharmaceutically acceptable means approved by a regulatory agency of the Federal or a state government or listed in the U.S. Pharmacopeia or other generally recognized pharmacopeia for use in animals, and more particularly in humans.

The process comprises injecting, under pressure, a solution containing the polynucleotides into the interior of a bone. In one embodiment, one component of the surgical device used consists of rigid or semi-rigid hollow tube or tubular conduit possessing a conical blunt end as illustrated in FIG. 2. The conical blunt end is shaped to fit into a hole that is formed in one wall of the target bone. The hole can be any shape that permits insertion of an injection device. In one embodiment, a conical shaped hole, as a countersink, is bored into the bone. An appropriate material for forming the rigid hollow tube, called the bone fitting, is stainless steel. The bone fitting is shaped and sized to fit snuggly into the hole. The fitting is also shaped and sized to enter into the interior space of the bone without touching the opposite interior wall of the bone. The fitting may further contain a gasket to ensure a sufficiently tight fit into the hole created in the bone. The gasket is a means to provide a seal against leakage during the injection. The gasket can be made from any surgically acceptable material that enables the formation of a seal. A particularly contemplated material is an elastic or semi-elastic material such as TYGON® tubing. Thus, the bone fitting allows a solution to be injected into the interior of the bone under pressure. The bone fitting can be designed to provide the further function of penetrating the bone, as in a trocar.

The bone fitting is connected to a reservoir. The reservoir contains the solution containing the polynucleotides that are to be delivered to the bone cells. The reservoir can be any container which allows a predetermined volume to be dispensed at a predetermined rate. Exemplary reservoirs include syringes or syringes connected to syringe pumps. The reservoir may be connected directly to the bone fitting or it may be connected to the bone fitting via a tube. The tube may be rigid, semi-rigid, or flexible. Any conventional IV tubing may be connected to the fitting.

The bone fitting is pressed into the bone with sufficient force to enable the solution to be injected into the bone under pressure. The fitting may be held in place with a clamp or vise. The fitting may be formed to readily enable it to be held by the clamp or vise. Alternatively, the fitting may contain a threaded end thereby allowing the fitting to be screwed into the bone compact tissue. The threads can be designed to aid in forming a bore in the bone or they can be dimensioned to fit tightly into a pre-formed hole in the bone.

An example of a bone injection system, including the bone fitting, optional gasket, reservoir and optional tubing, is illustrated in FIG. 2. The drawing illustrates a rigid stainless steal bone fitting A with a blunt conical tip A. The conical tip is fitted into an elastic surrounding D. The bone fitting is held by a microvise, C. The bone fitting is further connected to a reservoir by means of flexible tubing B. The reservoir consists of a syringe connected to a Harvard syringe pump (PHD 2000) which was used for the injection. The stainless steel bone fitting is pressed into a hole created in the bone wall. The gasket seals the space between the stainless steel tubing and the bone tissue, preventing leakage. The sizes of the conical tip with gasket and the hole allow the fitting to advance into the bone lumen without reaching opposite internal bone surface. The shape of the fitting and gasket, and the material of its construction, provides a means by which to direct pressure against the bone thereby enabling injection under pressure. All materials are chosen to be compatible with biological systems and reagents and suitable for surgical use.

Other devices which enable injection, under pressure, of fluid into the interior of a bone are compatible with the disclosed method of delivery of polynucleotides to bone cells.

Nearly any needle or similar device that can be used for standard intraosseous infusion can be used or modified to be used for gene delivery to bone using the described invention. In addition to standard injection devices, such as trocars, butterfly needles, spinal needles with stylet, and bone marrow biopsy needles, specific injection devices have been designed for intraosseous injection. These devices are typically designed to position an object a predetermined distance through the bone compact tissue and into the bone lumen. These specialized injection devices include straight-needle Jamshidi needles (Baxter), Diekmann (Cook Critical Care), SurFast (Cook Critical Care), Sussmane-Raszynski (Cook Critical Care), spring-loaded auto-injectors, BIG (Waismed), and F.A.S.T.1 (Pyng Medical Corporation). Because of the increased pressure used for delivery of polynucleotides to extravascular cells, the injection device must provide a means for sealing the injection point against leakage during the injection.

Non-viral nucleic acid—For the purposes of this invention, non-viral nucleic acid means nucleic acid that is not encapsulated within an intact viral coat. The term naked polynucleotide indicates that the polynucleotide is not associated with a transfection reagent or other delivery vehicle that is required for the nucleic acid or polynucleotide to be delivered to the cell.

It is predicted that the described methods may be combined with other delivery vehicles or vectors or other delivery enhancing groups. Such delivery vehicles and groups comprise: transfection reagents, viral vectors, non-viral vectors, lipids, polymers, polycations, amphipathic compounds, targeting signals, nuclear targeting signals, and membrane active compounds. The composition of the injection solution can depend on the nature of the molecule or complex that is to be delivered. Certain complexes may be delivered more efficiently using low salt injection solutions. The use of hypertonic or hypotonic injection solutions or the use of vasodilators in the injection solution may further enhance delivery.

Delivery of a gene to a cell that expresses a protein not previously expressed in the mammal can result in the induction of an immune response directed against the newly expressed protein. Also, the polynucleotide itself, or other potential components of the injection solution, may illicit an immune response. Therefore it may be beneficial to provide immunosuppressive drugs to the mammal. Suppression of immune response to an expressed gene can prolong expression of the gene. Immunosuppressive drugs can be given before, during, or after injection of the polynucleotide. Immunosuppression can be of short term duration (less than 3 months) or long term duration.

A therapeutic effect of the protein in attenuating or preventing the disease state can be accomplished by the protein either staying within the cell, remaining attached to the cell in the membrane or being secreted and dissociating from the cell where it can enter the general circulation and blood. Proteins on the membrane can have a therapeutic effect by providing a receptor for the cell to take up a protein or lipoprotein. For example, the low density lipoprotein (LDL) receptor could be expressed in hepatocytes and lower blood cholesterol levels and thereby prevent atherosclerotic lesions that can cause strokes or myocardial infarction. Therapeutic proteins that stay within the cell can be enzymes that clear a circulating toxic metabolite as in phenylketonuria. They can also cause a cancer cell to be less proliferative or cancerous (e.g. less metastatic). A protein within a cell could also interfere with the replication of a virus.

We have disclosed gene expression achieved from reporter genes in specific tissues. Levels of a gene product, including reporter (marker) gene products, are measured which then indicate a reasonable expectation of similar amounts of gene expression by transfecting other polynucleotides. Levels of treatment considered beneficial by a person having ordinary skill in the art differ from disease to disease, for example: Hemophilia A and B are caused by deficiencies of the X-linked clotting factors VIII and IX, respectively. Their clinical course is greatly influenced by the percentage of normal serum levels of factor VIII or IX: <2%, severe; 2-5%, moderate; and 5-30% mild. Thus, an increase from 1% to 2% of the normal level of circulating factor in severe patients can be considered beneficial. Levels greater than 6% prevent spontaneous bleeds but not those secondary to surgery or injury. A person having ordinary skill in the art of gene therapy would reasonably anticipate beneficial levels of expression of a gene specific for a disease based upon sufficient levels of marker gene results. In the hemophilia example, if marker genes were expressed to yield a protein at a level comparable in volume to 2% of the normal level of factor VIII, it can be reasonably expected that the gene coding for factor VIII would also be expressed at similar levels. Thus, reporter or marker genes such as the genes for luciferase and β-galactosidase serve as useful paradigms for expression of intracellular proteins in general. Similarly, reporter or marker genes secreted alkaline phosphatase (SEAP) serve as useful paradigms for secreted proteins in general.

The term polynucleotide, or nucleic acid or polynucleic acid, is a term of art that refers to a polymer containing at least two nucleotides. Nucleotides are the monomeric units of polynucleotide polymers. Polynucleotides with less than 120 monomeric units are often called oligonucleotides. Natural nucleic acids have a deoxyribose- or ribose-phosphate backbone. An artificial or synthetic polynucleotide is any polynucleotide that is polymerized in vitro or in a cell free system and contains the same or similar bases but may contain a backbone of a type other than the natural ribose-phosphate backbone. These backbones include: PNAs (peptide nucleic acids), phosphorothioates, phosphorodiamidates, morpholinos, and other variants of the phosphate backbone of native nucleic acids. Bases include purines and pyrimidines, which further include the natural compounds adenine, thymine, guanine, cytosine, uracil, inosine, and natural analogs. Synthetic derivatives of purines and pyrimidines include, but are not limited to, modifications which place new reactive groups such as, but not limited to, amines, alcohols, thiols, carboxylates, and alkylhalides. The term base encompasses any of the known base analogs of DNA and RNA. The term polynucleotide includes deoxyribonucleic acid (DNA) and ribonucleic acid (RNA) and combinations of DNA, RNA and other natural and synthetic nucleotides.

A polynucleotide can be delivered to a cell to express an exogenous nucleotide sequence, to inhibit, eliminate, augment, or alter expression of an endogenous nucleotide sequence, or to affect a specific physiological characteristic not naturally associated with the cell. Altering gene expression may comprise: altering splicing of an RNA, affecting mRNA levels, and altering gene expression through binding to transcription factors. A polynucleotides can also alter the sequence of a polynucleotide in a cell. This would include polynucleotides that alter the sequence of chromosomal DNA, cellular RNA, viral DNA, viral RNA. Altering the sequence of a polynucleotide in a cell includes altering the sequence through gene conversion or recombination. Chimeroplasts (hybrid molecules of RNA and DNA) and single stranded polynucleotides have been used to alter chromosomal DNA sequences.

A polynucleotide-based gene expression inhibitor comprises any polynucleotide containing a sequence whose presence or transcription in a cell causes sequence-specific degradation or inhibition of the function, transcription, or translation of a gene. Polynucleotide-based expression inhibitors may be selected from the group comprising: siRNA, microRNA, interfering RNA or RNAi, dsRNA, ribozymes, antisense polynucleotides, and DNA expression cassettes encoding siRNA, microRNA, dsRNA, ribozymes or antisense nucleic acids. SiRNA comprises a double stranded structure typically containing 15-50 base pairs and preferably 19-25 base pairs and having a nucleotide sequence identical or nearly identical to an expressed target gene or RNA within the cell. An siRNA may be composed of two annealed polynucleotides or a single polynucleotide that forms a hairpin structure. MicroRNAs (mRNAs) are small noncoding polynucleotides, about 22 nucleotides long, that direct destruction or translational repression of their mRNA targets. Antisense polynucleotides comprise sequence that is complimentary to a gene or mRNA. Antisense polynucleotides include, but are not limited to: morpholinos, 2′-O-methyl polynucleotides, DNA, RNA and the like. The polynucleotide-based expression inhibitor may be polymerized in vitro, recombinant, contain chimeric sequences, or derivatives of these groups. The polynucleotide-based expression inhibitor may contain ribonucleotides, deoxyribonucleotides, synthetic nucleotides, or any suitable combination such that the target RNA and/or gene is inhibited.

The term expression cassette refers to a natural or recombinantly produced nucleic acid molecule that is capable of expressing a gene or genetic sequence in a cell. An expression cassette typically includes a promoter (allowing transcription initiation), and a sequence encoding one or more proteins or RNAs. Optionally, the expression cassette may include transcriptional enhancers, non-coding sequences, splicing signals, transcription termination signals, and polyadenylation signals. An RNA expression cassette typically includes a translation initiation codon (allowing translation initiation), and a sequence encoding one or more proteins. Optionally, the expression cassette may include translation termination signals, a polyadenosine sequence, internal ribosome entry sites (IRES), and non-coding sequences. Optionally, the expression cassette may include a gene or partial gene sequence that is not translated into a protein. The nucleic acid can effect a change in the DNA or RNA sequence of the target cell. This can be achieved by hybridization, multi-strand nucleic acid formation, homologous recombination, gene conversion, RNA interference or other yet to be described mechanisms.

The term gene generally refers to a nucleic acid sequence that comprises coding sequences necessary for the production of a nucleic acid (e.g., siRNA) or a polypeptide or precursor. A polypeptide can be encoded by a full length coding sequence or by any portion of the coding sequence so long as the desired activity or functional properties (e.g., enzymatic activity, ligand binding, signal transduction) of the full-length polypeptide or fragment are retained. The term also encompasses the coding region of a gene and the including sequences located adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as 5′ untranslated sequences. The sequences that are located 3′ or downstream of the coding region and which are present on the mRNA are referred to as 3′ untranslated sequences. The term gene encompasses synthetic, recombinant, cDNA and genomic forms of a gene. A genomic form or clone of a gene contains the coding region interrupted with non-coding sequences termed introns, intervening regions or intervening sequences. Introns are segments of a gene which are transcribed into nuclear RNA. Introns may contain regulatory elements such as enhancers. Introns are removed or spliced out from the nuclear or primary transcript; introns therefore are absent in the mature RNA transcript. Components of a gene also include, but are not limited to, promoters, enhancers, transcription factor binding sites, polyadenylation signals, internal ribosome entry sites, silencers, insulating sequences, matrix attachment regions. Non-coding sequences influence the level or rate of transcription and/or translation of the gene. Covalent modification of a gene may influence the rate of transcription (e.g., methylation of genomic DNA), the stability of mRNA (e.g., length of the 3′ polyadenosine tail), rate of translation (e.g., 5′ cap), nucleic acid repair, nuclear transport, and immunogenicity. Gene expression can be regulated at many stages in the process. Up-regulation or activation refers to regulation that increases the production of gene expression products (i.e., RNA or protein), while down-regulation or repression refers to regulation that decrease production. Molecules (e.g., transcription factors) that are involved in up-regulation or down-regulation are often called activators and repressors, respectively.

It may be desirable to regulate expression of the delivered polynucleotide using regulated promoters. Regulated promoters may be inducible or repressible. Regulated gene expression systems may be selected from the list comprising: drug-dependent gene regulation, tetracycline/doxycycline-inducible, tetracycline/doxycycline-repressible, rapamycin-inducible, β-galactoside, streptogramin-regulated, bacterial repressor protein, antiprogestin-inducible GeneSwitch® (Valentis, Inc., induced by mifepristone), nuclear hormone receptor ligand binding domain (antiprogestin-, antiestrogen-, ecdysteroid-, glucocorticoid-responsive), heterodimeric protein, metabolic regulated, hypoxia responsive, and glucose responsive systems. Some of these systems are regulated by proteins naturally occurring in mammalian cells while others require co-delivery of a gene encoding a transcription activator or repressor. It may also be desirable for the delivered polynucleotide to be expressed from a bone cell specific promoter, such as a promoter active in osteoclasts, osteoblasts, stromal cells or hematopoietic cells.

Bone marrow stromal cells, including the primitive pluripotent mesenchymal stem cells (MSC) and multipotent adult progenitor cells (MAPC) are attractive targets for cell and gene therapy because they give rise to differentiated cells belonging to osteogenic, chondrogenic, adipogenic, myogenic and fibroblastic lineages. The polynucleotides can also be delivered to osteoblasts, osteoclasts, fibroblasts, osteogenic progenitors, and bone marrow cells. Genes encoding osteogenic factors may also be delivered to muscle cells or myoblasts, fibroblasts and mesenchymal stem cells surrounding the bone.

A number of genes have been proposed as being potentially relevant for bone gene therapy. These include anti-arthritic genes, osteoinductive cytokines, bone morphogenic proteins (BMP-2, BMP-3, BMP-4, BMP-6, BMP-7/osteogenic protein 1, BMP-9,), Calponin (basic or h1), Growth Hormone, Transforming growth factor β1, Tumor necrosis factor-α, purine nucleoside phosphorylase (LPNSN-2), IL-1 Receptor antagonist, Osteoprotegrin, Proalpha2(I) collagen, PTH, LIM mineralization protein-1 (LMP-1), Fibroblast growth factor, Platelet-derived growth factor, and Insulin-like growth factors. Various recombinant growth factors and bone morphogenetic proteins (BMPs) have proven to be potent stimulators of osteoinduction and have been used to heal bone defects in a variety of animal models, including rats, rabbits, dogs and non-human primates.

Several genes that encode different growth factors are involved in the normal proliferation and differentiation of osteoblasts in bone formation. Transcription factors related to osteoblast development (cbfa-1), cytokines, growth factors and their receptors (TGFβ family including the bone morphogenetic proteins, IL-6, IL-1β, IGF-1 etc), and enzymes of metabolic pathways such as metalloproteinases and aromatase can be potential candidates for gene therapy.

A biologically active compound is a compound having the potential to react with biological components. More particularly, biologically active compounds utilized in this specification are designed to change the natural processes associated with a living cell. For purposes of this specification, a cellular natural process is a process that is associated with a cell before delivery of a biologically active compound. Biologically active compounds may be selected from the group comprising: pharmaceuticals, proteins, peptides, polypeptides, hormones, cytokines, antigens, viruses, oligonucleotides, nucleic acids, and synthetic polymers such as polypyroles could also be delivered.

The process of delivering a polynucleotide to a cell has been commonly termed transfection or the process of transfecting and also it has been termed transformation. The term transfecting as used herein refers to the introduction of a polynucleotide or other biologically active compound into cells. The polynucleotide may be used for research purposes or to produce a change in a cell that can be therapeutic. The delivery of a polynucleotide for therapeutic purposes is commonly called gene therapy. The delivery of a polynucleotide can lead to modification of the genetic material present in the target cell. The term stable transfection or stably transfected generally refers to the introduction and integration of an exogenous polynucleotide into the genome of the transfected cell. The term stable transfectant refers to a cell which has stably integrated the polynucleotide into the genomic DNA. Stable transfection can also be obtained by using episomal vectors that are replicated during the eukaryotic cell division (e.g., plasmid DNA vectors containing a papilloma virus origin of replication, artificial chromosomes). The term transient transfection or transiently transfected refers to the introduction of a polynucleotide into a cell where the polynucleotide does not integrate into the genome of the transfected cell. If the polynucleotide contains an expressible gene, then the expression cassette is subject to the regulatory controls that govern the expression of endogenous genes in the chromosomes. The term transient transfectant refers to a cell which has taken up a polynucleotide but has not integrated the polynucleotide into its genomic DNA.

A transfection agent, or transfection reagent or delivery vehicle, is a compound or compounds that bind(s) to or complex(es) with oligonucleotides and polynucleotides, and enhances their entry into cells. Examples of transfection reagents include, but are not limited to, cationic liposomes and lipids, polyamines, calcium phosphate precipitates, histone proteins, polyethylenimine, polylysine, and polyampholyte complexes. It has been shown that cationic proteins like histones and protamines, or synthetic polymers like polylysine, polyarginine, polyornithine, DEAE dextran, polybrene, and polyethylenimine may be effective intracellular in vitro delivery agents. Typically, the transfection reagent has a component with a net positive charge that binds to the oligonucleotide's or polynucleotide's negative charge. For delivery in vivo, complexes made with sub-neutralizing amounts of cationic transfection agent may be preferred. Non-viral vectors is include protein and polymer complexes (polyplexes), lipids and liposomes (lipoplexes), combinations of polymers and lipids (lipopolyplexes), and multilayered and recharged particles. Transfection agents may also condense nucleic acids. Transfection agents may also be used to associate functional groups with a polynucleotide. Functional groups include cell targeting signals, nuclear localization signals, compounds that enhance release of contents from endosomes or other intracellular vesicles (such as membrane active compounds), and other compounds that alter the behavior or interactions of the compound or complex to which they are attached (interaction modifiers).

The cell targeting signal can be cell receptor ligands, such as proteins, peptides, sugars, steroids and synthetic ligands as well as groups that interact with cell membranes, such as lipids, fatty acids, cholesterol, dansyl compounds, and amphotericin derivatives. The signal may increase binding of a compound to the cell surface and/or its association with an intracellular compartment. Other targeting groups can be used to increase the delivery of the polynucleotide to certain parts of the cell, such as nuclear localization signals.

### EXAMPLES

### Example 1

The delivery device consists of stainless steel tubing (bone fitting, outer diameter 0.81 mm) 30 mm in length with a flat tip ground into a conical shape (FIG. 2). The stainless steel tubing was connected to Teflon tubing (internal diameter 0.7 mm) and gripped in a microvise, with the tip extending out of the microvise by 10 mm. The tip was fitted into a TYGON® tubing gasket (internal diameter 0.5 mm) and cut into a conical shape. This delivery devise was connected to a Harvard syringe pump (PHD 2000) which was used for the injection.

Animals were anesthetized with isoflurane and positioned in an anterior recumbent position. A skin incision was made on the lateral surface of the leg over the femur. Using skin/muscle retractors and cotton swabs, the middle-proximal surface of the femur was exposed. A hole was drilled into the exposed femur using a 0.78 mm microbit and Proxxon rotary tool. The conical tip of the stainless steel tubing with attached Tygon tubing gasket was inserted into the hole. When the tip of stainless steel tubing was pressed into the hole, the Tygon tubing gasket sealed the space between the stainless steel tubing bone fitting and the bone tissue, preventing any leakage. The sizes of the conical tip with Tygon tubing and hole allowed the stainless steel tubing to advance into the bone lumen 0.2-0.3 mm without reaching opposite internal bone surface.

Several plasmid DNAs were injected using this method: pLuc (containing a luciferase expression cassette), pSEAP(containing a secreted alkaline phosphatase expression cassette), pLacZ (containing a β-galactosidase expression cassette), and pEYFP (containing a yellow fluorescent protein expression cassette) were tested. Each gene was under the control of the CMV promoter.

For delivery to rat femur, 1.1 to 2.5 ml was injected at a rate of 0.66 ml/second. For delivery to mouse femur, 0.2 ml was injected as a rate of 0.66 ml/second. No short- nor long-term complications or pathology was observed to be associated with the injection.

For luciferase expression analysis, soft tissues (muscles and ligaments) were removed from the femur and the bone marrow was expelled from bone. The adjacent muscle, bone and bone marrow were each analyzed separately.

For pSEAP expression analysis, blood samples were collected.

For EYFP expression analysis, bone decalcification with whole limb in situ perfusion with heparin/formalin/EDTA treatment was employed followed by tissue cryosectioning. To identify expressing cells in bone, sections were immunostained for collagen and CD45 antigen (leukocyte common antigen). Bone sections also were stained for DNA (ToPro3) and actin (Alexa 546).

Results:

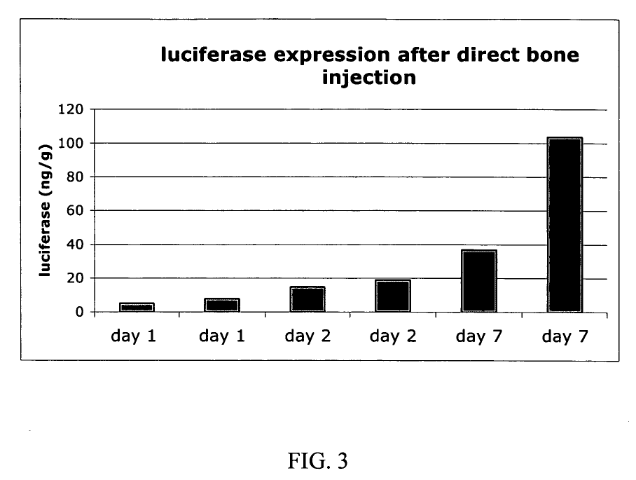

### Total Luciferase Expression—ICR Mice (FIG. 3)

| DAY | RLU | ng | ng/gm |
|---|---|---|---|
| day 1 | 122334 | 0.31 | 5.20 |
| day 1 | 185932 | 0.47 | 7.90 |
| day 2 | 351139 | 0.90 | 14.92 |
| day 2 | 445996 | 1.14 | 18.95 |
| day 7 | 865362 | 2.21 | 36.78 |
| day 7 | 2437326 | 6.22 | 103.59 |

### Anatomical Location of Expressing Cells (Proximal, Medial, and Distal Segments, Bone Marrow)—Sprague Dawley Rats

| | Rat 1 | | Rat 2 | | Rat 3 | |
|---|---|---|---|---|---|---|
| Luciferase (pg) | left | right | left | right | left | right |
| lymph node | 0.8 | 1.8 | 0.2 | 0.1 | 5.8 | 0.5 |
| distal bone | 5.3 | 11.3 | 3.9 | 1.6 | 6.0 | 6.3 |
| bone section w/hole | 7.7 | 6.0 | 4.5 | 11.4 | 5.1 | 4.1 |
| bone marrow | 0.9 | 0.6 | 0.5 | 2.3 | 0.4 | 0.3 |
| proximal bone | 1.9 | 340.6 | 12.1 | 5.1 | 436.0 | 330.9 |

| | | | |
|---|---|---|---|
| liver | 15.0 | 7.6 | 7.2 |
| kidney | 2.1 | 2.1 | 2.0 |
| right leg muscle | 150.6 | 2.4 | 3398.5 |
| left leg muscle | 7.8 | 7.0 | 256.3 |

### SEAP Expression—Sprague Dawley Rats

| SEAP (ng/ml) | day 0 | day 1 | day 3 | day 7 |
|---|---|---|---|---|
| rat 1 | 0.04 | 18.22 | 17.69 | 6.18 |
| rat 2 | 0.04 | 15.07 | 12.31 | 1.98 |
| average | 0.04 | 16.65 | 15.00 | 4.08 |

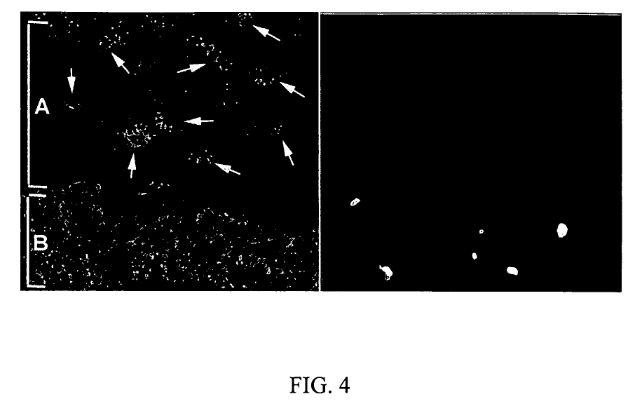

Yellow Fluorescent Protein expression results (Microscopic localization and type of cells expressing reporter gene). Most of the expressing cells appeared in the proximal segment of the femur. Fluorescent microscopy showed that almost all expressing cells appeared in the periosteum (FIG. 4).

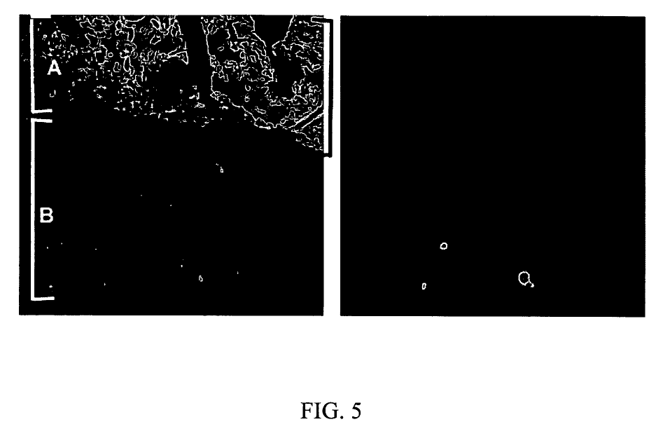

The percentage of expressing cells in periosteum varied from 1% to 10%, depending on area. Periosteum location of expressing cells was confirmed with collagen immunostaining of bone sections (FIG. 5). The compact bone accumulates collagen at high density (FIG. 5, Left-A), while periosteum accumulates less collagen (FIG. 5, Left-B). The majority of YFP-expressing cells appeared in the periosteum (FIG. 5, Right). The number of expressing cells in compact bone and in/or association with bone marrow tissue was much lower and inconsistent.

As a delivery site, the lateral surface of the middle-proximal region of a femur was used. Because the lateral surface of the middle-proximal region of a femur was exposed without bleeding, it was assumed that vasculature integrity of the area was preserved. After injection, a swelling of femoral muscles was immediately observed in predominantly deep muscles of the medial femoral region. To comprehend the fate of the injected fluid, the following experimental and anatomical observations were taking into account: 1) a lack of appearance of a free fluid coming out of bone, muscles, or into the surgical incision; 2) an injectant volume exceeded bone internal volume about 10 times; 3) muscle swelling was almost simultaneous with injection; and 4) a rigidity of a compact bone. Taking in account these constrains, it was concluded that: 1) compact femur bone served as a rigid tubular conductor for fluid; and 2) the first targeted area of the delivery was bone periost, a less dense bone compound with extensive vasculature that drains blood into periosseus venous plexus.

The foregoing is considered as illustrative only of the principles of the invention. Furthermore, since numerous modifications and changes will readily occur to those skilled in the art, it is not desired to limit the invention to the exact construction and operation shown and described. Therefore, all suitable modifications and equivalents fall within the scope of the invention.

We claim:

1. A method for delivering a polynucleotide to a bone cell in a mammal comprising: injecting into the lumen of said bone a solution containing said polynucleotide.

2. The method of claim 1 wherein the solution is injected under pressure.

3. The method of claim 1 wherein the bone cell is selected from the group consisting of: osteocyte, osteoclast, osteoblast, osteogenic progenitor cell, bone marrow cell, mesenchymal stem cell, multipotent adult progenitor cell, and stromal cells.

4. The process of claim 1 wherein the cell is located in periosteum tissue of said bone.

5. The process of claim 1 wherein injecting said solution results in increased permeability of veins in said bone.

6. The process of claim 1 wherein the polynucleotide is a naked polynucleotide.

7. The process of claim 1 wherein the polynucleotide comprises of an gene expression vector.

8. The process of claim 1 wherein the polynucleotide comprises RNA.

9. The process of claim 1 wherein the polynucleotide comprises DNA.

10. The process of claim 1 wherein the polynucleotide is selected from the group consisting of siRNA, mRNA, mRNA and antisense RNA.

11. The process of claim 1 wherein the polynucleotide is associated with a transfection reagent.

12. The process of claim 1 wherein the polynucleotide encodes an osteogenic factor.

13. The process of claim 12 wherein the osteogenic factor consists of a bone morphogenic protein.
